(19) United States
(12) Patent Application Publication
(10) Pub. No.: US 2025/0058081 A1
(43) Pub. Date: Feb. 20, 2025

(54) Urinary Placement Catheter for Intravesical Devices

(71) Applicant: TARIS Biomedical LLC, Lexington, MA (US)

(72) Inventors: Anthony DiUbaldi, Lexington, MA (US); James Fleming, Lexington, MA (US); Hong Linh Ho Duc, Lexington, MA (US); Wayne Johnson, Louisville, KY (US); Mark Lewis, Lexington, MA (US); Jason Mello, Tyngsboro, MA (US)

(21) Appl. No.: 18/721,021

(22) PCT Filed: Dec. 20, 2022
(86) PCT No.: PCT/US2022/053460

§ 371 Date: Jun. 17, 2024

Related U.S. Application Data
(60) Provisional application No. 63/292,063, filed on Dec. 21, 2021.

(51) Int. Cl. A61M 25/00

(52) U.S. Cl. CPC A61M 25/0068; A61M 2025/0034; A61M 2210/1085

(57) ABSTRACT

An inserter assembly (200) for deploying an intravesical device into a urinary bladder of a patient includes a placement catheter (210), a receiving funnel (220), and a stylet (230). The placement catheter defines a lumen extending from a proximal catheter end to an exit opening. The receiving funnel is secured to the placement catheter. The stylet is removably received through the receiving funnel within the lumen. The stylet includes a stylet body and a paddle (238). The stylet body is dimensioned to drive the intravesical device through the lumen and the exit opening into the bladder. The paddle is fixed to the stylet body about the proximal stylet end and is engageable with the receiving funnel. The paddle optionally includes a fitting (242) being lockably engageable with the receiving funnel to removably secure the stylet to the placement catheter.

### CROSS-REFERENCE TO RELATED APPLICATIONS

This application claims priority to and benefit of U.S. Provisional Patent Application No. 63/292,063, filed Dec. 21, 2021, which is incorporated herein by reference.

### BACKGROUND

### Technical Field

This disclosure is generally in the field of medical devices for introduction into the body of a patient, and more particularly in the field of inserter devices and methods for deploying intravesical devices through the urethra and into the urinary bladder.

### Description of Related Prior Art

Intravesical devices may be used to deliver drugs or sense conditions within the bladder. Devices and systems for placement of such devices into a patient's bladder are described, for example, in U.S. Pat. No. 8,721,621, which is incorporated by reference herein.

### BRIEF SUMMARY

In one aspect, an inserter assembly is provided for deploying an intravesical device into a urinary bladder of a patient, wherein the inserter assembly includes a placement catheter defining a lumen, the lumen extending from a proximal catheter end to an exit opening; a receiving funnel secured to the placement catheter at the proximal catheter end; and a stylet removably received through the receiving funnel within the lumen of the placement catheter, wherein the stylet comprises (i) a stylet body extending between a proximal stylet end and a distal stylet end, the stylet body being dimensioned to drive the intravesical device through the lumen and the exit opening into the bladder, and (ii) a paddle fixed to the stylet body about the proximal stylet end, wherein the paddle is engageable with the receiving funnel.

In another aspect, an inserter assembly is provided for deploying an intravesical device into a urinary bladder of a patient, wherein the inserter assembly includes a placement catheter defining a lumen, the lumen extending from a proximal catheter end to an exit opening; and a stylet removably received through the lumen of the placement catheter, wherein the stylet comprises a stylet body extending between a proximal stylet end and a distal stylet end, the stylet body being dimensioned to drive the intravesical device through the lumen and the exit opening into the bladder, and a paddle fixed to the stylet body about the proximal stylet end, wherein the paddle comprises a central body portion surrounding the proximal stylet end, a thumb rest defined at a proximal paddle end, and a pair of wing portions extending laterally from the central body between the thumb rest and a distal paddle end.

In yet another aspect, a method is provided for deploying an intravesical device in a bladder using an inserter assembly including a placement catheter and a stylet, wherein the method includes (i) introducing the intravesical device in a lumen of the placement catheter through a receiving funnel of the inserter assembly, wherein an exit opening of the deployment body is present in an interior volume of the bladder; (ii) introducing a distal stylet end of the stylet in the lumen of the placement catheter through the receiving funnel to contact the intravesical device within the lumen; and (iii) distally advancing the stylet through the receiving funnel to drive the intravesical device along the lumen toward the exit opening and to release the intravesical device through the exit opening into the bladder.

### BRIEF DESCRIPTION OF THE DRAWINGS

Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1A is a plan view of an embodiment of an inserter assembly including a placement catheter, a receiving funnel, and a stylet.

FIG. 1B is a cross-sectional view of the placement catheter shown in FIG. 1A, taken along line A-A.

FIG. 2A is a front view of a placement catheter of an inserter assembly, according to an embodiment of the present disclosure.

FIG. 2B is a side view of the inserter assembly of FIG. 2A.

FIG. 3A is a perspective view of a hub portion of an inserter assembly, including a proximal end portion of a placement catheter, a receiving funnel, and a stylet, according to an embodiment of the present disclosure.

FIG. 3B is a top end view of the inserter assembly of FIG. 3A.

FIG. 3C is a plan view of the proximal portion of the inserter assembly of FIG. 3A.

FIG. 4A is a perspective view of a proximal portion of an inserter assembly, including a proximal end portion of a placement catheter with hub having an asymmetric receiving funnel, and a stylet, according to another embodiment of the present disclosure.

FIG. 4B is a plan view of a proximal portion of the inserter assembly of FIG. 4A in a first configuration, in which the stylet is partially inserted into the placement catheter.

FIG. 4C is a plan view of the proximal portion of the inserter assembly of FIG. 4A in a second configuration, in which the stylet is fully inserted into the placement catheter.

FIG. 4D is a partial cross-sectional view of the proximal portion of the inserter assembly of FIG. 4C, showing snap fit engagement between the hub/funnel and a fitting on the paddle of the stylet.

FIGS. 5A and 5B are views illustrating manual manipulation of the proximal portion of the inserter assembly shown in FIGS. 4A to 4D.

FIGS. 6A and 6B are side and top views, respectively, of a proximal portion of an inserter assembly including hub having an asymmetric receiving funnel and a stylet having a paddle with a curved thumb rest at its end, according to an embodiment of the present disclosure.

FIGS. 7A and 7B are side and top views, respectively, of a proximal portion of an inserter assembly including a hub having a symmetric receiving funnel and a stylet having a paddle with a flat end.

FIG. 8 is a partial side view of an embodiment of an inserter assembly including a placement catheter and a stylet without a fitting, and without a receiving funnel.

FIG. 9 is a block diagram of an embodiment of a method of deploying an intravesical device within a patient's bladder.

### DETAILED DESCRIPTION

It would be desirable to provide improved inserter devices that provides better, more secure user handling of the inserter device components, that facilitates handling of a lubricant used with the inserter device, that provides better cooperation between the luminal body and the stylet, that provides enhanced operational feedback, including certainty of intravesical device deployment, or a combination thereof.

Improved inserter devices have been developed for placement of intravesical devices into the bladder of a patient. The devices are assemblies of a placement catheter component and a stylet component that cooperate to release an intravesical device from a contained position within a lumen of the placement catheter to a deployed position outside of the placement catheter.

The intravesical device used with the presently disclosed inserter devices may be any medical device suitable for deployment within a patient's bladder. In some embodiments, the intravesical device is a drug delivery device. Examples of such drug delivery devices are described in U.S. Pat. Nos. 10,543,166, 9,283,361, 10,729,823, 10,315,019, 10,737,078, and 11,020,575, which are incorporated herein by reference. In some embodiments, the intravesical device includes diagnostic components, such as cameras and sensors. Examples of such devices are described in U.S. Pat. No. 11,065,426 and U.S. Publication No. 2021/0196124, which are incorporated herein by reference.

The terms “distal” or “distally” generally refer to a direction toward the patient, while the terms “proximal” or “proximally” generally refer to a direction away from the patient and toward a clinician. The patient may be a human in need of medical diagnosis, treatment, or prophylaxis.

The present inserter assembly includes a placement catheter, a receiving funnel, and a stylet. The placement catheter defines a lumen extending from a proximal catheter end to an exit opening. The exit opening is at or near a distal end of the placement catheter. The intravesical device may be advanced through the lumen to the exit opening and into the bladder, driven by the stylet.

The stylet is configured to drive one or more intravesical devices through the lumen of the placement catheter into the bladder. For example, an intravesical device may be present or introduced in the lumen, and the stylet may be introduced into the lumen to drive the intravesical device along the lumen. In particular, a distal end of the stylet pushes against a surface, such as an end, of the intravesical device to displace the intravesical device within the lumen toward and through an exit opening in the placement catheter.

The receiving funnel includes a hub that is secured to the placement catheter at the proximal catheter end. The receiving funnel may facilitate introducing the stylet into a proximal end of the catheter, for example, without needing precise alignment between the catheter and the stylet. For example, a tip or end of the stylet may be guided along an interior of the funnel toward a proximal opening and into a lumen of the catheter. Likewise, the receiving funnel also may facilitate introducing the intravesical device into the lumen of the catheter, for example, prior to introduction of the stylet. Further, the receiving funnel beneficially may aid the clinician in introducing a lubricant into the lumen, without requiring use of a syringe to direct the lubricant into the catheter lumen. For example, precisely locating the fine or narrow opening of the lumen may not be required to introduce the lubricant, because the funnel interior and surface would guide the lubricant to the opening.

The stylet is removably received through the receiving funnel within the lumen of the placement catheter. The stylet includes a stylet body extending between a proximal stylet end and a distal stylet end. The stylet body is dimensioned to drive the intravesical device through the lumen and the exit opening into the bladder.

The stylet further advantageously includes a paddle at the proximal stylet end to aid secure handling and manipulation of the stylet, which may particularly be needed when the clinician's gloved fingers may have lubricant on them. The paddle defines two opposed faces extending between paddle ends, for example, faces that can be grasped, pinched, or held. The surface of the paddle may include ridges or texture to facilitate a user's grip of it. The paddle is sometimes referred to as a “hub” of the stylet.

In some embodiments, the paddle includes a fitting that is configured to lockingly engage with the receiving funnel to removably secure the stylet to the placement catheter. The fitting may provide positive (tactile and/or audible) feedback to the clinician when the stylet is fully inserted into the catheter. For example, the lockably engageable fitting may include one or more mechanical features for snap-lock engagement with complementary mechanical features on the receiving funnel. In preferred embodiments, the lockable engagement feature can be manipulated to unlock and disengage the stylet (e.g., the fitting) from the placement catheter (e.g., the receiving funnel).

In some other embodiments, the paddle does not include a locking feature. The placement catheter and stylet may be configured such that during insertion of the stylet, the fitting of the stylet is engageable with the receiving funnel. For example, the fitting may be brought into stopping contact with an interior surface within the receiving funnel, without locking, wherein the stylet subsequently can be freely withdrawn from the placement catheter.

The stylet has a length sufficient to completely drive the intravesical device out through the exit opening when the stylet is fully inserted. Thus, the positive feedback also provides a positive indication that the intravesical device is deployed into the bladder. The engagement of the paddle with the funnel, or the optional lockable engagement of the fitting with the funnel, may also prevent over-insertion of the stylet relative to the catheter, which may prevent unintended introduction of the stylet into the interior of the bladder beyond the end of the catheter, or unintended contact of the stylet and a wall of the bladder. The engagement of the paddle with the funnel may take the form of a distal surface end of the paddle surrounding the stylet coming into interfacing contact with a proximal end surface of the placement catheter, which completes/stops the insertion.

An intravesical device may be placed in a patient's bladder using one of the inserter assemblies described herein. In some embodiments, the method includes introducing the intravesical device in an elongated deployment configuration in the lumen of the placement catheter through the receiving funnel. The exit opening is present in an interior volume of the bladder. The method further includes introducing a distal stylet end in the lumen of the placement catheter through the receiving funnel to contact the intravesical device within the lumen. The method further includes distally advancing the stylet through the receiving funnel to drive the intravesical device along the lumen toward the exit opening and to release the intravesical device through the exit opening into the bladder. The method optionally further includes engaging the paddle of the stylet with the receiving funnel, which may serve to indicate that the distal end of the stylet has reached a position has reached a position at/near the exit opening, to thereby indicate that the intravesical device necessarily has been released into the bladder. In some other embodiments, the paddle need not engage with the funnel for the intravesical device to be released from the placement catheter.

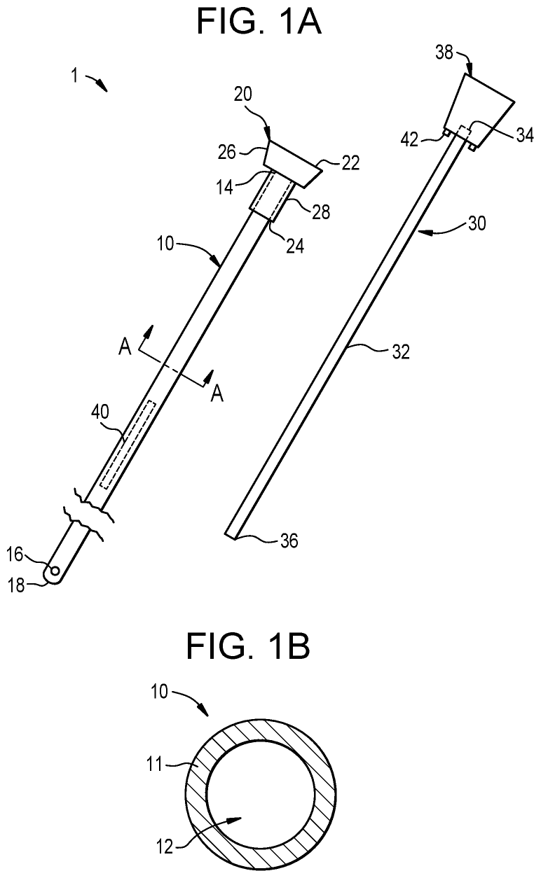

Embodiments of inserter assemblies are shown in FIGS. 1A through 8. Generally, the inserter assemblies include a placement catheter that is designed to pass through a patient's urethra, and a stylet introducible in the placement catheter.

FIG. 1A shows an inserter assembly 1 including a placement catheter 10, a receiving funnel 20, and a stylet 30. FIG. 1B shows the cross-section of the placement catheter 10 shown in FIG. 1A, taken along line A-A. The inserter assembly 1 may be used to deploy an intravesical device 40. An intravesical device 40 is shown disposed within a lumen of the placement catheter 10. The intravesical device 40 may or may not be initially present in the inserter assembly 1. For example, a clinician may introduce the intravesical device 40 into the placement catheter 10 of the inserter assembly 1 during or shortly before deployment. In some embodiments, the intravesical device 40 may be provided as part of the inserter assembly 1, for example, within the placement catheter 10, without needing introduction of the intravesical device 40 into the inserter assembly 1 by the clinician. The cross-sectional shape of the lumen 12 may match the cross-sectional shape of the intravesical device 40.

In some embodiments, the inserter assembly 1 includes the placement catheter 10, the receiving funnel 20, and the stylet 30. The placement catheter 10 defines a lumen 12 extending from a proximal catheter end 14 to an exit opening 16. In some embodiments, the exit opening 16 may be adjacent a distal catheter end 18. The exit opening 16 is in communication with the lumen 12 in a manner that facilitates smooth passage of the intravesical device 40 from the lumen 12 through the exit opening 16.

In some other embodiments, the exit opening may be at the distal end of the placement catheter and aligned with the lumen.

A wall 11 of the placement catheter 10 generally defines the lumen 12. The wall 11 is formed from a biocompatible polymeric material, with a thickness providing a column strength suited to prevent buckling of the wall while the placement catheter is being inserted through a urethra. The material preferably is amenable to conventional sterilization processes, such as gamma irradiation or ethylene oxide sterilization. In certain embodiments, the placement catheter 10 is transparent or translucent to visually determine the position of the stylet 30 and/or the intravesical device 40 within the lumen 12.

The lumen 12 may have a circular, elliptical, or oval cross-sectional profile, or any other suitable profile. The intravesical device 40 may be positioned within the lumen 12, ready for deployment. Thus, in certain embodiments, the intravesical device 40 may be preloaded within the lumen 12 of the placement catheter 10. For example, the intravesical device 40 may be preloaded or loaded by the clinician or an apparatus before use into the lumen 12 through the exit opening 16, or through a proximal opening of the lumen 12 through the receiving funnel 20, or in an alternative embodiment (not shown) through a slit along the wall 11.

Generally, the distal catheter end 18 and the distal portion of the placement catheter 10 is configured for traversing the urethra with no or minimal trauma to urethral tissues. The proximal catheter end portion 14 and a proximal portion of the placement catheter 10 remains outside of the body, providing access for a user, such as a medical professional.

The dimensions of the placement catheter 10 may be selected based at least in part on the dimensions of the anatomy that the inserter assembly 1 is designed to navigate. The placement catheter 10 may have any suitable length. In certain embodiments, the placement catheter 10 has a length of at least 10 cm, or of at least 20 cm, or of at least 30 cm, or of at least 40 cm, and less than 60 cm, or less than 80 cm. In certain embodiments, the placement catheter has a length of from 10 cm to 40 cm.

The placement catheter 10 may have any suitable outer diameter. In certain embodiments, the placement catheter has an outer diameter of from 5 Fr (1.67 mm outer diameter) to 18 Fr (6 mm outer diameter). In some other embodiments, the placement catheter may have an outer diameter that is outside the range of 5 to 18 Fr, for example 20, Fr, 22, Fr, 24, Fr, 26 Fr, 28 Fr, or 30 Fr.

One or both of the stylet 30 and the intravesical device 40 may be introduced into the lumen 12 of the placement catheter 10 via or through the receiving funnel 20. The receiving funnel 20 may have a shape which expands from a narrow end to a wide end. In some embodiments, the receiving funnel 20 may define an aperture adjacent the narrow end that is fluidically coupled to the lumen 12 of the placement catheter. The receiving funnel 20 may have a height that is shallower than one or both of a length or a width of the funnel. In some embodiments, the receiving funnel may have a height having a ratio to a width of from 0.25 to 0.5.

The receiving funnel 20 may be removably securable to the placement catheter 10 by any suitable coupling mechanism, for example, by one or more clips, friction fit, or a pressure sensitive adhesive. In certain embodiments, the receiving funnel 20 is formed of a material that is softer than the material of the placement catheter 10, such that the receiving funnel can be elastically or plastically pushed, deformed, or placed about the placement catheter 10. In certain embodiments, the receiving funnel 20 is permanently secured to the placement catheter 10, for example, by molding, overmolding, or permanent adhesive, or any suitable mechanism that secures the receiving funnel 20 the placement catheter 10 in a manner that cannot be removed nondestructively.

In certain embodiments, the receiving funnel 20 is overmolded about the placement catheter 10 at the proximal catheter end 14. In certain embodiments, the receiving funnel 20 is integral with the placement catheter 10 at the proximal catheter end 14. As used herein, the terms “integral with” and “integrally formed” refer to the funnel and placement catheter being part of the same monolithic structure/material, e.g., co-molded. The receiving funnel 20 may be secured to the placement catheter 10 at, adjacent, or about the proximal catheter end 14. In certain embodiments, the receiving funnel 20 is integrally formed with the placement catheter 10. For example, a rear or proximal portion of the placement catheter may be formed as the receiving funnel 20.

The receiving funnel 20 extends between a proximal funnel end 22 and a distal funnel end 24. The receiving funnel 20 may consist of a single or integral unit extending between the proximal funnel end 22 and a distal funnel end 24. In certain embodiments, two or more units may be separately formed and secured to form the receiving funnel 20. For example, the receiving funnel 20 may include a funnel portion 26 and a hub 28 that can be integrally formed as a single unit, or may be separately formed and secured to each other. The hub 28 may be dimensioned to be secured to or about the proximal catheter end 14 of the placement catheter 10. The funnel portion 26 may be dimensioned to widen in a proximal direction away from the proximal catheter end 14 to facilitate receiving the stylet 30, the intravesical device 40, or a fluid, such as a lubricant, within the lumen 12 of the placement catheter 10.

In the embodiment shown in FIG. 1A, the narrowest width of the funnel portion 26 is wider than the width of the hub 28. In certain embodiments, the width of the hub 28 may increase to smoothly transition into a width of the funnel portion 26. Thus, the exterior surface of the funnel portion 26 may smoothly extend into the exterior surface of the hub 28.

The receiving funnel 20 may have any suitable profile, for example, a profile defined by a curve, a contour, a polygon, a linear or piecewise linear path, or combinations thereof. In certain embodiments, the receiving funnel 20 has a circular or oval profile. In certain embodiments, the receiving funnel 20 is flattened, such that the receiving funnel 20 has a shorter minor axis and a wider major axis in a direction transverse to the receiving funnel 20. In certain embodiments, the receiving funnel is dimensioned to receive at least 0.1 mL, or at least 0.5 mL, or at least 1 mL of a liquid or gel, e.g., a lubricant.

The funnel portion 26 defines a funnel volume fluidically coupled to the lumen 12 of the placement catheter 10. Thus, the receiving funnel 20 may be used to guide components or fluids into the lumen 12 without requiring an exact determination of the location or alignment of a proximal opening of the lumen 12. The proximal catheter end 14, as shown in FIG. 1A, may extend to and align with an interface of the funnel portion 26 and the hub 28. Thus, a proximal opening of the lumen 12 may align with that interface. In certain embodiments, the proximal catheter end 14 may not extend to that interface, and may instead, terminate at a position along the hub 28.

In certain embodiments, the receiving funnel 20 is soft and elastically deformable, for example, in response to a force exerted by the clinician. In other embodiments, the receiving funnel 20 is rigid, and substantially retains its shape in response to a force exerted by the clinician.

A proximal portion of the stylet 30 adjacent the proximal stylet end 34 remains outside of the patient's body and is configured for receiving a driving force from a clinician or an apparatus. A distal portion adjacent the distal stylet end 36 is configured for driving the intravesical device 40 from the exit opening 16.

The stylet 30 is removably received through the receiving funnel 20 within the lumen 12 of the placement catheter 10. The stylet 30 includes a stylet body 32 extending between a proximal stylet end 34 and a distal stylet end 36. The stylet body 32 is dimensioned to drive the intravesical device 40 through the lumen 12 and the exit opening 16 into the patient's bladder.

The overall shape and configuration of the stylet 30 permits applying the driving force to the intravesical device 40 without substantial friction between the placement catheter 10 and the stylet 30 and without the stylet 30 causing trauma to the placement catheter 10 or to the intravesical device 40.

The dimensions of the stylet 30 may be selected based at least in part on the dimensions of the placement catheter 10 lumen and the size of the intravesical device. The stylet 30 may be slightly longer than the placement catheter 10. Once inserted into the deployment instrument, the stylet proximal end 34 remains outside of the proximal catheter end 14 of the placement catheter 10, and the distal stylet end 36 reaches to the distal catheter end 18 of the placement catheter 10. Such a configuration permits inserting the stylet 30 along the full length of the placement catheter 10 to drive the intravesical device 40 from the exit opening 16.

An outer diameter of the stylet 30 is smaller than an inner diameter of the placement catheter 10, so that the stylet 30 can be inserted into the deployment instrument without having to overcome the force of friction along the length of the entire placement catheter 10. In some embodiments, the stylet body has an outer diameter that is about 1 mm less than the inner diameter of the lumen of the placement catheter.

The stylet 30 is formed from a suitable material that has a column strength suited to resist buckling during insertion into the placement catheter lumen and driving of the intravesical device. The material may be a biocompatible polymer, such as silicone, polyethylene, or polyurethane and preferably is amenable to conventional sterilization procedures. The stylet 30 can be formed, for example, by extrusion or molding processes known in the art.

In the illustrated embodiment, the stylet 30 has a substantially cylindrical outer surface, although other configurations are possible.

In certain embodiments, the stylet body 32 has a solid cross-section along an entirety of the stylet body 32. In other embodiments, the stylet body 32 may be hollow. For example, the stylet body 32 may define a lumen extending from the proximal stylet end 34 to the distal stylet end 36. In some such embodiments, the lumen of the stylet body 32 may be used to permit passage of fluids into or out of the placement catheter lumen and/or the bladder. In some embodiments, the lumen may extend through the paddle.

The stylet 30 further includes a paddle 38 at, adjacent, or about the proximal stylet end 34. The paddle 38 is engageable with the receiving funnel 20, for example, by contact between surfaces of the paddle 38 and the receiving funnel 20. Optionally, the paddle 38 is lockably engageable with the receiving funnel 20 to removably secure the stylet 30 to the receiving funnel 30 and ultimately to the placement catheter 10.

The paddle 38 optionally includes at least one fitting 42 for promoting lockable engagement of the paddle 38 to the funnel 20. The fitting 42 may include at least one clip, for example, one, two or more clips. In certain embodiments, the fitting 42 includes at least one clip removably securable to the receiving funnel 30. For example, the at least one clip may be securable to a surface or a feature of the receiving funnel 30, for example, a lip, a rim, a contour, a protrusion, a channel, a ridge, an opening, or any suitable feature complementary to the at least one clip (or generally to the fitting 42). In certain embodiments, the fitting 42 is removably securable to or at an interior surface of the receiving funnel 20, for example, adjacent the proximal catheter end 14. The fitting 42 may exert a biasing or securing force such that once stylet 30 is fully engaged and secured to the receiving funnel 20 (and thus to the placement catheter 10), the stylet 30 will remain secured to the receiving funnel 20 until at least a minimum threshold removing force is exerted on the stylet 30 in a proximal direction away from the placement catheter 10.

In certain embodiments, the fitting 42 generates a tactile or audible feedback upon engagement. In certain embodiments, the stylet 30 itself is configured to generate audible or tactile feedback during engagement with the receiving funnel 20, for example, by snapping or tapping against the receiving funnel 20. Thus, the fitting 42 may facilitate a complete engagement and securement of the stylet 30 to the placement catheter 10, until intentionally released by the clinician.

Instead of, or in addition to fitting 42 including a clip, the inserter assembly 1 may include other mechanisms for securing the stylet 30 to the placement catheter 10. For example, one of the fitting 42 or the receiving funnel 20 may define a circumferential ridge, and the other of the fitting 42 or the receiving funnel 20 may define a circumferential channel dimensioned to matingly engage the circumferential ridge. The mating ridge and channel may be suitably elastic and dimensioned to provide a “snap-fit” locking engagement.

In some other embodiments, the fitting 42 and the receiving funnel 20 may be securable by a luer lock or a twist-lock.

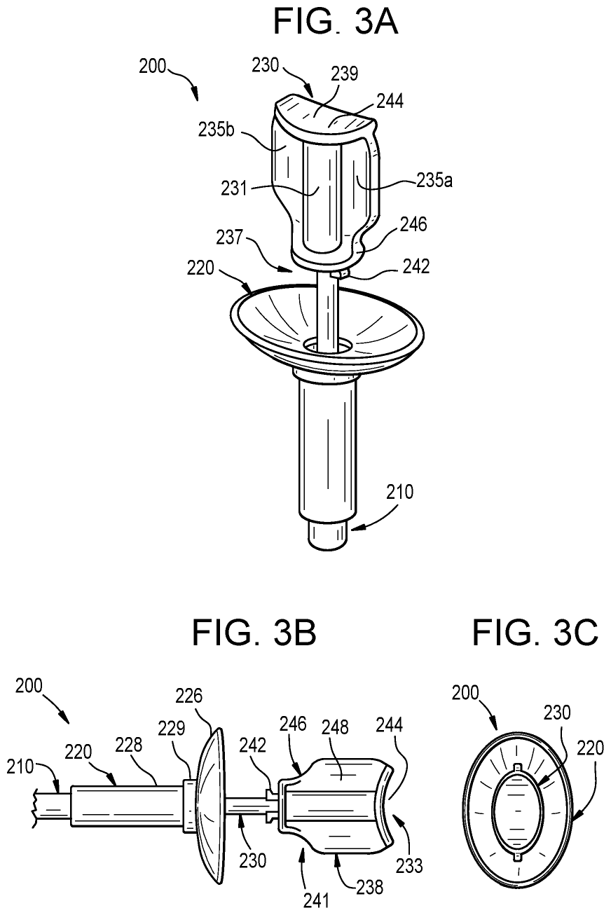

While in the embodiment of FIG. 1A, the fitting 42 is associated with the stylet 30, in other embodiments, a fitting may be provided on one or both of the stylet 30 (for example, at the paddle 38) and the receiving funnel 20, securable to the other of the stylet 30 (or the paddle 38) and the receiving funnel 20. In certain embodiments, the fitting 42 includes two clips distally extending from a distal paddle end and securable to an interior funnel lip. Further description of the fitting and funnel engagement are described below with reference to FIGS. 3A to 7B.

In certain other embodiments, an inserter assembly may not include a fitting, as described below with reference to FIG. 8.

The paddle 38 may be secured to the stylet body 32, for example, by a friction or interference fit, molding, overmolding, or permanent adhesive, or any suitable mechanism that secures the paddle 38 to the stylet body 32, for example, in a manner that cannot be removed by a clinician. The proximal stylet end 34 may extend to and align with an end of the paddle 38. Thus, the proximal ends of the stylet body 32 and the paddle 38 may be flush or aligned. In certain embodiments, the proximal stylet end 34 may not extend in such a manner and instead may terminate at a position within an interior of the paddle 38, as shown in FIG. 1A. In certain embodiments, the paddle 38 is overmolded about the stylet body 32 at the proximal stylet end 34. In certain embodiments, the paddle 38 may be coupled to the stylet body by an adapter. In certain embodiments, the paddle 38 is integral with the stylet body 32. For example, the stylet body 32 and the paddle 38 may be molded together, or as a single unit or piece.

The paddle 38 is dimensioned to engage with the receiving funnel 20. For example, the paddle 38 may be wider or larger than the receiving funnel 20 in at least one dimension, such that the paddle 38 may be secured at or about an exterior surface, rim, or lip of the receiving funnel 20. In certain embodiments, the paddle 38 is narrower or smaller than the receiving funnel 20 in at least one dimension, such that the paddle 38 is at least partially received within and engaged against an interior surface of the funnel portion 26. In certain such embodiments, a portion of the paddle 38 may extend proximally outward the funnel portion 26, such that a clinician may grasp, pinch, or grab the extending portion and exert a pulling force on the paddle 38 to disengage or remove the paddle 38 (and thus the stylet 30) from the receiving funnel 20. In certain embodiments, no portion of the paddle 38 extends out from the receiving funnel 20, so that the clinician cannot easily manually remove or disengage the paddle 38 (and thus the stylet 30), for example, without using a tool such as a forceps. Such a configuration may be used to prevent or avoid unintentional disengagement or removal of the stylet 30 from the placement catheter 10.

In certain embodiments, the placement catheter 10 defines a rounded atraumatic tip at the distal catheter end 18, for example, as shown in FIG. 1A. In other embodiments, the distal end of the placement catheter 10 may have a relatively flat or tapered tip so long as suitably configured to permit insertion into/through the urethra.

One or more of the placement catheter 10, the paddle 38, the receiving funnel 20, and the stylet 1 may be formed of the same or different polymeric material. In certain embodiments, the inserter assembly 1 includes a radiomarker. For example, the placement catheter 10 or the stylet body 32 may include a radio-opaque material or marker.

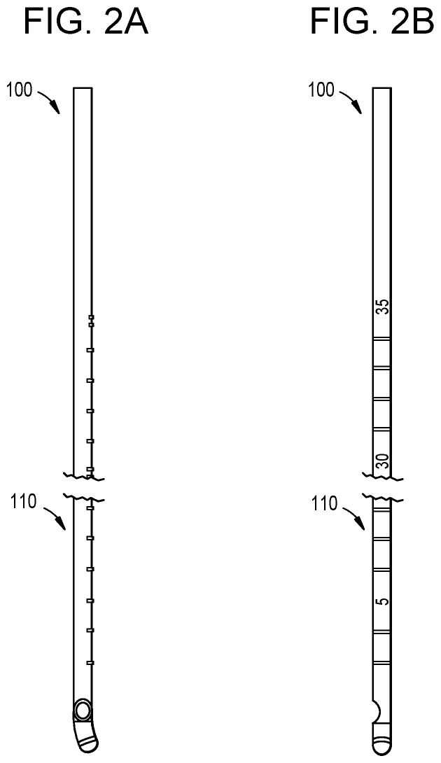

The inserter assembly 1 may be used to drive and deploy an intravesical device 40. Further embodiments of inserter assemblies or components thereof are described with reference to FIGS. 2A to 8, with similarly numbered reference numerals designating like elements. It will be understood that the inserter assembly 1 may include components modified according to such and other embodiments.

FIGS. 2A-2B illustrate one embodiment of a placement catheter 110 of an inserter assembly 100. The placement catheter 110 has a rounded atraumatic tip including a coudé bend. When a coudé bend is present, the coudé bend may be aligned at a predetermined angle relative (for example, aligned with, or normal to) a major axis of the retrieval funnel such that an orientation of the retrieval funnel is indicative of an orientation of the coudé bend. In certain embodiments, the placement catheter 110 may include depth markings indicative of insertion depth and exit opening orientation, as shown in FIG. 2B.

One preferred embodiment of an inserter assembly 200 is shown in FIGS. 3A-3C. The placement catheter 210 may be generally similar to the placement catheter 10 described with reference to FIGS. 1A-1B or the placement catheter 110 described with reference to FIGS. 2A-2B. The funnel portion 226 of the receiving funnel 220 has an oval profile, as seen in FIGS. 3B and 3C. A rim 229 may be present at an external interface of the funnel portion 226 and the hub 228.

Stylet 230 includes a paddle 238 including a fitting 242. The fitting 242 includes two clips, extending distally from the fitting 238. The two clips of the fitting 242 are received within and removably securable to an interior of the receiving funnel 220.

The paddle 238 has a central body portion 231 surrounding the proximal stylet end, a thumb rest 244 defined at a proximal paddle end 233, and a pair of wing portions 235a, 235b extending laterally from the central body portion 231 between the thumb rest 244 and a distal paddle end 237. The thumb rest 244 has a concave surface 239 facing away from the central body portion 231 and an oval shaped periphery, the longer axis of which extends the same direction as the wing portions extend from the central body portion. Each of the wing portions 235a, 235b includes a taper 241 leading to the distal paddle end 237. The paddle 238 also includes a curved finger rest 246 proximate to each taper 241. Surface areas of the wing portions 235a, 235b optionally include a grip surface 248 extending between the proximal paddle end and the distal paddle end. For example, the grip surface may include a non-slip coating material known in the art or a texturized area (e.g., a plurality of ridges, grooves, raised bumps, or other surface roughening features). One or more of the thumb rest 244, curved finger rest 246, and the grip surface 248 may facilitate holding, grasping, grabbing, pulling, and pushing of the stylet 230 relative to the placement catheter 210 and the receiving funnel 220.

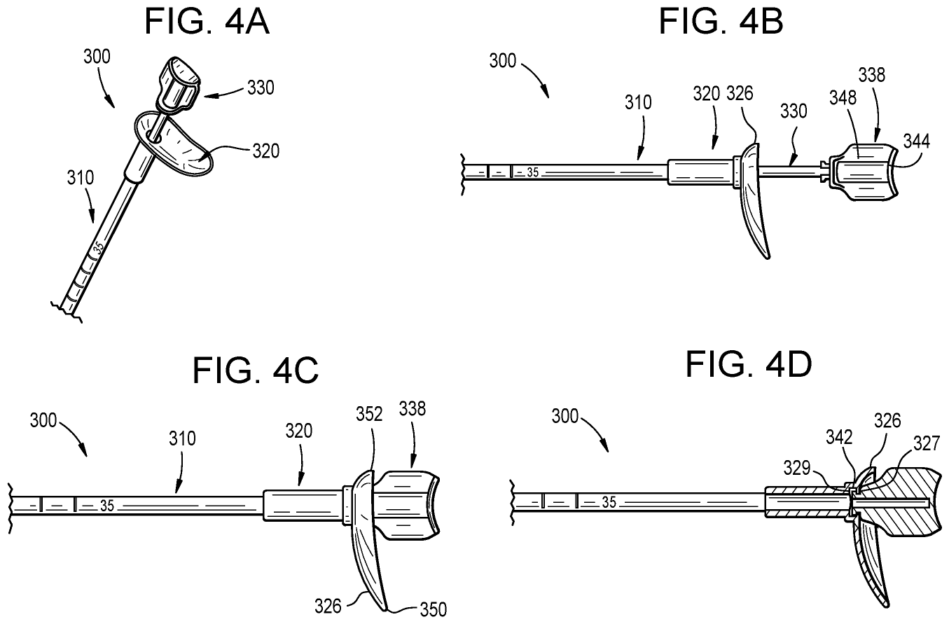

Another preferred embodiment of an inserter assembly 300 is shown in FIGS. 4A-4D. The inserter assembly 300 is substantially similar to the inserter assembly 200 described with reference to FIGS. 3A to 3C, but with the receiving funnel 320 extending asymmetrically in a direction transverse to the stylet body 330.

As shown in FIG. 4A the inserter assembly 300 includes a placement catheter 310, an asymmetric receiving funnel 320, and a stylet 330. In a first illustrated configuration, the stylet 330 is partially inserted into placement catheter 310, as shown in FIGS. 4A-4B. In a second illustrated configuration, the stylet 330 is fully inserted into the lumen of the placement catheter 310, as shown in FIGS. 4C-4D.

The placement catheter 310 includes depth markings, similar to the placement catheter 110 shown in FIGS. 2A-2B. A first funnel edge 350 of the funnel portion 326 of the receiving funnel 320 is proximally offset and extends further proximally relative a second funnel edge 352, as shown in FIG. 4C. The paddle 338 includes the thumb rest 344 and the gripping surface 348, as shown in FIG. 4B.

The paddle 338 includes a fitting 342 including two clips securable to the inner rim 327 in an interior of the funnel portion 326, and received within a channel 329 adjacent the inner rim 327, as shown in FIG. 4D.

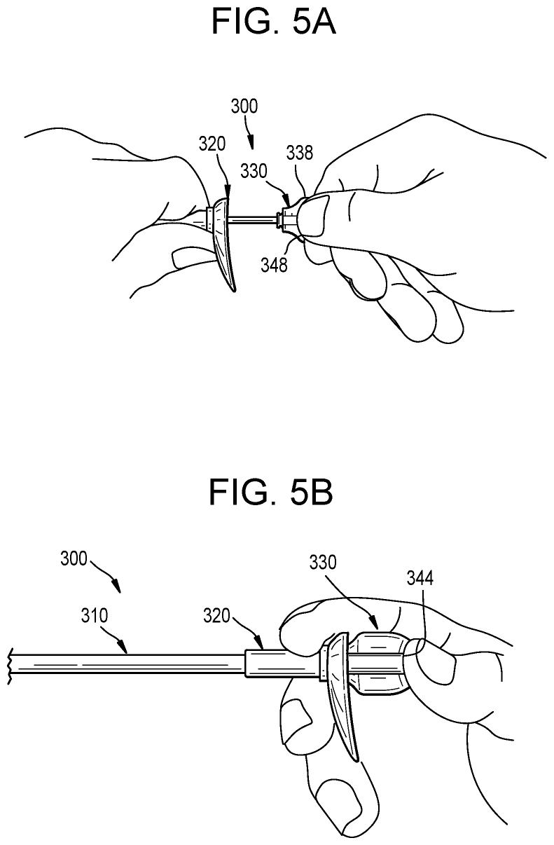

The asymmetric configuration of the receiving funnel 320 of the inserter assembly 300 may facilitate ergonomical manual operation of the inserter assembly 300, as shown in FIGS. 5A-5B. FIG. 5A is a view illustrating a use of the inserter assembly 300 of FIG. 4A with the stylet 330 partially inserted through the placement catheter 310. FIG. 5B is a view illustrating a use of the inserter assembly 300 of FIG. 4A with the stylet 330 fully inserted. As shown in FIG. 5A, the thumb of one hand of a user can rest against the asymmetric portion of the receiving funnel 320, with the thumb and fingers of the user's other hand gripping the grip surface 348 of the paddle 338. This configuration can be used to either advance or retract the stylet 330 distally or proximally relative to the funnel 320. To completely insert the stylet 330, the thumb of one hand can exert a final push on the curbed thumb rest 344 of the stylet 330, with fingers resting against the receiving funnel 320, as shown in FIG. 5B.

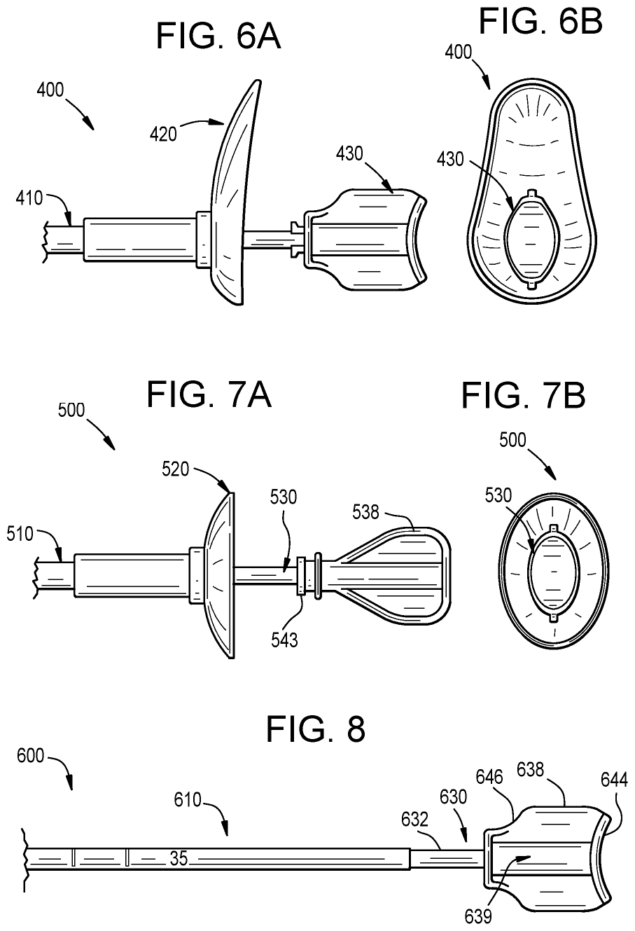

Another preferred embodiment of an inserter assembly 400 is shown in FIGS. 6A-6B. The inserter assembly 400 includes a placement catheter 410, an asymmetric receiving funnel 420, and a stylet 430. The inserter assembly 400 is substantially similar to the inserter assembly 300 described with reference to FIGS. 4A-4D but with the receiving funnel 420 having a pear-shaped asymmetric profile in a direction transverse to the stylet body 430. The shape of the receiving funnel 420 may facilitating holding of the asymmetric funnel 420, for example, between fingers or between fingers and thumb.

Another preferred embodiment of an inserter assembly 500 is shown in FIGS. 7A-7B. The inserter assembly 500 including a placement catheter 510, a symmetric receiving funnel 520, and a stylet 530. The inserter assembly 500 is substantially similar to the inserter assembly 200 described with reference to FIGS. 3A-3C but with the paddle 538 of the inserter assembly 500 having a different shape than the paddle 238 of the inserter assembly 200. Paddle 538 has a tapered shape similar to a guitar pick, and also includes a twist-lock 543 (e.g., Luer lock) securable within an interior of receiving funnel 520.

FIG. 8 illustrates an alternative embodiment of an inserter assembly 600 that includes a placement catheter 610 and a stylet 630 having neither a fitting nor a receiving funnel. Apart from the absence of the fitting or the receiving funnel, the inserter assembly 600 is substantially similar to the inserter assembly 200 described with reference to FIGS. 3A to 3C. The stylet 630 includes a stylet body 632 extending between a proximal stylet end and a distal stylet end. The stylet 630 includes a paddle 638 fixed to the stylet body 632 about the proximal stylet end. The paddle 638 defines a curved thumb rest 644 at a proximal paddle end, and a curved finger rest 646 at a distal paddle end. The curved thumb 30) rest 644 may extend along a direction transverse to the stylet body 632, for example, perpendicular to the stylet body 632, or within ±10° of a direction perpendicular to the stylet body 632. In embodiments, the curved finger rest 646 is a first curved finger rest, and the paddle 638 further defines a second curved finger rest at the distal paddle end, as shown in FIG. 8.

The paddle 638 is fixed to the stylet body 632. For example, the paddle 638 may be overmolded about the stylet body 632 at the proximal stylet end. In such embodiments, the paddle 638 has a central body portion 639 defining a paddle lumen in which a proximal end portion of the stylet body 632 extends. In an alternative embodiments, the paddle 638 is integrally formed with the stylet body 632, such as in an injection molding process.

In some embodiments, the inserter assembly 600 may not include a receiving funnel, for example as shown in FIG. 8. In such embodiments, the paddle 638 may engage with the placement catheter 610. For example, a distal portion of the paddle 638 may contact or engage with a proximal portion of the placement catheter 610. Thus, a clinician may stop driving or pushing the stylet body 632 upon receiving audio or tactile feedback generated by engagement of the paddle 638 with the placement catheter 610. In other embodiments, the inserter assembly 600 may include a receiving funnel according to an embodiment of the disclosure, and the paddle 638 may engage with the receiving funnel.

While embodiments of insertion assemblies including a receiving funnel, a paddle, and a locking feature or fitting have been described, in other embodiments, the insertion assemblies may include one or two of a receiving funnel, a paddle, and a locking feature or fitting. In some embodiments, one or both of a receiving funnel and the fitting may be absent from inserter assemblies. In embodiments in which the receiving funnel is absent, the paddle may engage with or rest against a proximal end of the placement catheter after advancing the stylet through the placement catheter.

Various insertion assemblies according to the present disclosure may be provided as respective kits. For example, the kit may include a package that holds a placement catheter and a stylet. The kit may include a separate hub with integral receiving funnel, securable to the placement catheter prior to use, or the receiving funnel may be bonded or secured to the placement catheter. The kit may include an intravesical device to be introduced within the placement catheter, or may include the intravesical device preloaded within the placement catheter.

In certain embodiments, the deployment assemblies may be housed in at least one package, or kit, that stores one or more of the placement catheter, the receiving funnel, the stylet, and the intravesical device. The package protects the packaged components before the insertion procedure. For example, the components may be sterilized together, and transported together, in the package. One or both of the stylet or the receiving funnel can be either packaged with the placement catheter, packaged separately, or omitted completely. Regardless of which components are packaged together, the package is sterilized, such as using gamma irradiation or ethylene oxide sterilization.

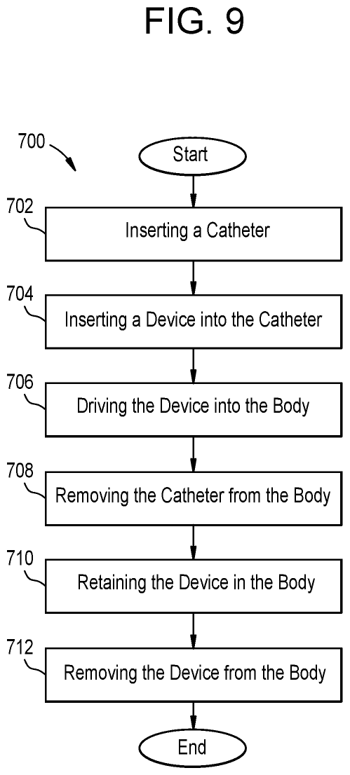

FIG. 9 is a block diagram of an embodiment of a method of inserting an intravesical device in the bladder. The method of FIG. 9 may be practiced using any insertion assembly according to the present disclosure, for example, an insertion assembly including a placement catheter, a receiving funnel, and a stylet.

In block 702, a placement catheter is inserted into the body. Inserting the placement catheter generally includes inserting the placement catheter into the urethra and driving the placement catheter forward until a distal end is positioned in the bladder, while a proximal end remains outside of the body. For example, the distal stylet end may be introduced in the lumen of the placement catheter through the receiving funnel to contact the intravesical device within the lumen.

In some embodiments, the placement catheter is inserted into the body in block 702 without the use of any additional devices. In some other embodiments, the placement catheter is inserted into the body in block 702 in association with a cystoscope, which permits visualizing the insertion procedure.

In some embodiments, inserting the placement catheter into the body in block 702 further includes verifying a distal end of the placement catheter has become positioned in the bladder. For example, the location of the distal end can be verified by communicating urine through the placement catheter from the distal end positioned in the bladder and/or by using the depth indicator markings (e.g., numbers) on the placement catheter. Alternatively, the location of the distal end may be verified by visualizing the distal end of the placement catheter with a cystoscope, an ultrasound or x-ray.

In embodiments in which the placement catheter includes a coudé bend, or tip, the clinician may rotate the placement catheter, and in turn the coudé bend, to control the orientation at which the intravesical device exits the placement catheter. For example, the coudé bend may orient the exit opening towards a direction that can be controlled by rotating the placement catheter. Thus, in some embodiments, the clinician to may orient, steer, or direct the intravesical device away from any anatomical features in the bladder that could impede release of the intravesical device from placement catheter.

In block 704, the intravesical device is inserted into the placement catheter. In some cases, inserting the intravesical device into the placement catheter can include elastically deforming the intravesical device from a non-linear bladder retention shape (e.g., a coil shape or an S-shape) to a substantially linear deployment shape. For example, block 704 may include introducing the intravesical device in an elongated deployment configuration in the lumen of the placement catheter through the receiving funnel, wherein the exit opening is present in an interior volume of the bladder.

Inserting the intravesical device into the placement catheter may include inserting the intravesical device into the distal catheter end, into the proximal catheter end, or into an opening or slit along the wall of the placement catheter. In some cases, inserting the intravesical device into the placement catheter comprises pre-loading the intravesical device into the placement catheter before the placement catheter is inserted into the body. In such cases, the order of blocks 702 and 704 is reversed. The intravesical device is typically pre-loaded if the device is placed in the placement catheter through the distal end or slit. although the intravesical device can be pre-loaded in any case for convenience. The intravesical device may be lubricated as, or before, the intravesical device is inserted into the placement catheter, to reduce friction associated with driving the intravesical device through the placement catheter in block 706.

In block 706, the intravesical device is driven into the bladder. In embodiments in which the intravesical device is inserted into the placement catheter through the receiving funnel into the proximal catheter end in block 704, driving the intravesical device into the bladder includes driving the intravesical device from the proximal catheter end, along the lumen, and through the distal catheter end until the intravesical device exits the placement catheter into the bladder. In embodiments in which the intravesical device is inserted into the distal catheter end in block 704, driving the intravesical device into the bladder includes driving the intravesical device through the distal catheter end until the intravesical device exits the placement catheter into the bladder, as the intravesical device was previously driven through the urethra upon insertion of the placement catheter in block 702.

Driving the intravesical device into the bladder in block 706 includes driving the intravesical device with a stylet. The stylet may be an embodiment of the stylet described above. In such cases, driving the intravesical device into the bladder in block 706 includes inserting the distal stylet end into the proximal catheter end portion through the receiving funnel and advancing the stylet through the placement catheter until the distal stylet end reaches the distal catheter end. Driving the intravesical device into the bladder in block 706 includes contacting the intravesical device with the stylet, advancing the intravesical device through the placement catheter using the stylet, and driving the intravesical device from the placement catheter with the stylet. For example, the stylet may be distally advanced through the receiving funnel to drive the intravesical device along the lumen toward the exit opening, and the fitting of the stylet or a portion of the paddle may optionally be engaged with the receiving funnel as an indication of complete stylet insertion and release of the intravesical device through the exit opening into the bladder.

In some embodiments in which the method is used to place an intravesical device that is configurable between a deployment shape (e.g., a linear shape) and a retention shape (e.g., a non-linear shape), the act of driving the intravesical device from the placement catheter in block 706 removes the force of the placement catheter wall from the intravesical device. This unloading may permit the intravesical device to elastically return to its retention shape for retaining the intravesical device within the bladder during a medically indicated (e.g., treatment or diagnostic) period. In other words, the driving may remove a restraint or confinement imposed on the intravesical device by the placement catheter wall.

In some embodiments, snap lock engagement of the fitting to the receiving funnel provides tactile and/or audible feedback that the stylet is fully inserted, which thereby ensures that the intravesical device necessarily has been deployed. In some embodiments, another portion of the paddle besides the fitting may contact the funnel, providing tactile and/or audible feedback of stylet insertion. For example, the In some embodiments, driving the intravesical device into the bladder in block 706 may further include observing the intravesical device in the bladder to ensure the device was properly deployed. For example, the device may be observed using a cystoscope, ultrasound, or x-ray.

In some embodiments, driving the intravesical device into the bladder in block 706 further includes introducing a volume of fluid into the bladder before the device is driven into the bladder. The fluid may be, for example, water or saline. The fluid may serve as a cushion in the bladder, reducing the likelihood of the intravesical device contacting the posterior of the bladder upon deployment, which may reduce bladder discomfort.

In block 708, the placement catheter and stylet are removed from the body. The stylet can be removed be either before or simultaneously with the placement catheter. For example, the paddle may be released from the receiving funnel, and the stylet proximally withdrawn through the lumen and from the catheter. In other embodiments, the paddle may remain engaged with the receiving funnel and the stylet within the lumen during the withdrawing of the catheter from the bladder. Thereafter, in block 710, the intravesical device remains deployed within the bladder for a period as medically indicated. In embodiments in which the intravesical device is a drug delivery device, the device is retained in the body in block 710 to release a drug into the body, such as to release drug from the device into the bladder and to the urothelial tissues and other local or regional tissues.

In some embodiments, the method includes removing the intravesical device from the body in block 712. Removing the intravesical device from the body may include inserting a removal instrument into the body, locating the deployed intravesical device in the body, grasping or otherwise securing the intravesical device, and pulling the intravesical device from the body using the removal instrument. The removal instrument may include use of a cystoscope or catheter that is inserted through the urethra until its distal end reaches the bladder, and forceps or other means may be used to facilitate grasping or securing the intravesical device.

Some embodiments of the present disclosure can be described in view of one or more of the following:

Embodiment 1. An inserter assembly for deploying an intravesical device into a urinary bladder of a patient, the inserter assembly comprising: (i) a placement catheter defining a lumen, the lumen extending from a proximal catheter end to an exit opening; (ii) a receiving funnel secured to the placement catheter at the proximal catheter end; and (iii) a stylet removably received through the receiving funnel within the lumen of the placement catheter, wherein the stylet comprises (a) a stylet body extending between a proximal stylet end and a distal stylet end, the stylet body being dimensioned to drive the intravesical device through the lumen and the exit opening into the bladder, and (b) a paddle fixed to the stylet body about the proximal stylet end, wherein the paddle is engageable with the receiving funnel.

Embodiment 2. The inserter assembly of Embodiment 1, wherein the paddle is overmolded about the stylet body at the proximal stylet end.

Embodiment 3. The inserter assembly of Embodiment 1 or 2, wherein the paddle is integral with the stylet body.

Embodiment 4. The inserter assembly of any one of Embodiments 1 to 3, wherein the paddle defines a curved thumb rest at a proximal end.

Embodiment 5. The inserter assembly of Embodiment 4, wherein the paddle further defines a curved finger rest opposing the curved thumb rest.

Embodiment 6. The inserter assembly of any one of Embodiments 1 to 5, wherein the paddle comprises a grip surface.

Embodiment 7. The inserter assembly of any one of Embodiments 1 to 6, wherein the paddle is dimensioned to be received within the receiving funnel.

Embodiment 8. The inserter assembly of any one of Embodiments 1 to 7, wherein the paddle comprises a fitting being lockably engageable with the receiving funnel to removably secure the stylet to the placement catheter.

Embodiment 9. The inserter assembly of Embodiment 8, wherein the fitting is engageable with an interior surface of the receiving funnel.

Embodiment 10. The inserter assembly of Embodiments 8 or 9, wherein one or both of the fitting and the receiving funnel comprises at least one clip securable to the other of the fitting and the receiving funnel.

Embodiment 11. The inserter assembly of Embodiment 10, wherein the fitting comprises two clips distally extending from a distal paddle end and securable to an interior funnel lip.

Embodiment 12. The inserter assembly of any one of Embodiments 8 to 11, wherein the fitting defines a circumferential ridge, and wherein the receiving funnel defines a circumferential channel dimensioned to matingly engage the circumferential ridge.

Embodiment 13. The inserter assembly of any one of Embodiments 8 to 12, wherein the fitting is configured to generate audible and/or tactile feedback during engagement with the receiving funnel.

Embodiment 14. The inserter assembly of any one of Embodiments 1 to 13, wherein the receiving funnel is overmolded about the proximal catheter end.

Embodiment 15. The inserter assembly of any one of Embodiments 1 to 14, wherein the receiving funnel is integral with the placement catheter at the proximal catheter end.

Embodiment 16. The inserter assembly of any one of Embodiments 1 to 15, wherein the receiving funnel has one or both of an oval profile and an oval perimeter.

Embodiment 17. The inserter assembly of any one of Embodiments 1 to 16, wherein the receiving funnel extends asymmetrically in a direction transverse to the stylet body

Embodiment 18. The inserter assembly of any one of Embodiments 1 to 17, wherein the receiving funnel defines a funnel lumen configured to transfer lubricant from the receiving funnel to the lumen of the placement catheter.

Embodiment 19. The inserter assembly of Embodiment 18, wherein the receiving funnel is dimensioned to receive at least 1 mL of the lubricant.

Embodiment 20. The inserter assembly of Embodiments 18 or 19, wherein the receiving funnel is shaped to facilitate transfer of the lubricant from a packet into the lumen of the placement catheter.

Embodiment 21. The inserter assembly of any one of Embodiments 1 to 20, wherein the stylet body has a solid cross-section along an entirety of the stylet body.

Embodiment 22. The inserter assembly of any one of Embodiments 1 to 21, wherein the placement catheter is transparent or translucent to visually determine the position of the intravesical device or the stylet within the lumen.

Embodiment 23. The inserter assembly of any one of Embodiments 1 to 22, wherein a distal catheter end of the placement catheter comprises a rounded atraumatic tip.

Embodiment 24. The inserter assembly of any one of Embodiments 1 to 23, wherein a distal catheter end of the placement catheter comprises a coudé bend.

Embodiment 25. The inserter assembly of Embodiment 24, wherein the coudé bend is aligned with a major axis of the retrieval funnel such that an orientation of the retrieval funnel is indicative of an orientation of the coudé bend.

Embodiment 26. The inserter assembly of any one of Embodiments 1 to 25, wherein the placement catheter comprises depth markings indicative of insertion depth and exit opening orientation.

Embodiment 27. The inserter assembly of any one of Embodiments 1 to 26, wherein the placement catheter has a length of from 10 cm to 40 cm.

Embodiment 28. The inserter assembly of any one of Embodiments 1 to 27, wherein the placement catheter has an outer diameter of from 5 Fr (1.67 mm outer diameter) to 30 Fr (10 mm outer diameter).

Embodiment 29. The inserter assembly of any one of Embodiments 1 to 28, wherein one or both of the placement catheter and the stylet include a radiomarker.

Embodiment 30. The inserter assembly of any one of Embodiments 1 to 29, wherein one or more of the placement catheter, the paddle, the receiving funnel, and the stylet are formed of the same or different polymeric material.

Embodiment 31. The inserter assembly of any one of Embodiments 1 to 30, further comprising the intravesical device loaded within the lumen of the placement catheter.

Embodiment 32. The inserter assembly of any one of Embodiments 1 to 31, wherein the paddle comprises: a central body portion surrounding the proximal stylet end; a thumb rest defined at a proximal paddle end; and a pair of wing portions extending laterally from the central body portion between the thumb rest and a distal paddle end.

Embodiment 33. The inserter assembly of Embodiment 32, wherein the thumb rest has a concave surface facing away from the central body, an oval shaped periphery, or both a concave surface facing away from the central body and an oval shaped periphery.

Embodiment 34. The inserter assembly of Embodiment 32 or 33, wherein each of the wing portions comprises a taper leading to the distal paddle end.

Embodiment 35. The inserter assembly of Embodiment 34, wherein the paddle further comprises a curved finger rest proximate to each taper.

Embodiment 36. The inserter assembly of any one of Embodiments 32 to 35, wherein the paddle is overmolded about the stylet body at the proximal stylet end.

Embodiment 37. The inserter assembly of any one of Embodiments 32 to 35, wherein the paddle is integral with the stylet body.

Embodiment 38. An inserter assembly for deploying an intravesical device into a urinary bladder of a patient, the inserter assembly comprising: a placement catheter defining a lumen, the lumen extending from a proximal catheter end to an exit opening; and a stylet removably received through the lumen of the placement catheter, wherein the stylet comprises a stylet body extending between a proximal stylet end and a distal stylet end, the stylet body being dimensioned to drive the intravesical device through the lumen and the exit opening into the bladder, and a paddle fixed to the stylet body about the proximal stylet end, wherein the paddle comprises a central body portion surrounding the proximal stylet end, a thumb rest defined at a proximal paddle end, and a pair of wing portions extending laterally from the central body between the thumb rest and a distal paddle end.

Embodiment 39. The inserter assembly of Embodiment 38, wherein the thumb rest has a concave surface facing away from the central body, an oval shaped periphery, or both a concave surface facing away from the central body and an oval shaped periphery.

Embodiment 40. The inserter assembly of Embodiment 38 or 39, wherein each of the wing portions comprises a taper leading to the distal paddle end.

Embodiment 41. The inserter assembly of Embodiment 40, wherein the paddle further comprises a curved finger rest proximate to each taper.

Embodiment 42. The inserter assembly of any one of Embodiments 38 to 41, wherein the paddle is overmolded about the stylet body at the proximal stylet end.

Embodiment 43. The inserter assembly of any one of Embodiments 38 to 41, wherein the paddle is integral with the stylet body.

Embodiment 44. The inserter assembly of any one of Embodiments 38 to 43, further comprising a receiving funnel secured to the placement catheter at the proximal catheter end, wherein the stylet is removably received through the receiving funnel.

Embodiment 45. A method for deploying an intravesical device in a bladder of a patient using an inserter assembly including a placement catheter and a stylet, the method comprising: (i) introducing the intravesical device in a lumen of the placement catheter through a receiving funnel of the inserter assembly, wherein an exit opening of the deployment body is present in an interior volume of the bladder; (ii) introducing a distal stylet end of the stylet in the lumen of the placement catheter through the receiving funnel to contact the intravesical device within the lumen; and (iii) distally advancing the stylet through the receiving funnel to drive the intravesical device along the lumen toward the exit opening and to release the intravesical device through the exit opening into the bladder.

Embodiment 46. The method of Embodiment 45, further comprising stopping the distally advancing when a paddle of the stylet engages with the receiving funnel.

Embodiment 47. The method of Embodiment 45, further comprising stopping the distally advancing when a fitting of the stylet engages with the receiving funnel.

Embodiment 48. The method of Embodiment 47, wherein the fitting is part of a paddle of the stylet.

Embodiment 49. The method of any one of Embodiments 46 to 48, wherein the stopping comprises locking engagement between the stylet and the receiving funnel.

Embodiment 50. The method of any one of Embodiments 45 to 49, further comprising, before introducing the intravesical device into the lumen, transurethrally advancing a distal portion of the placement catheter into the bladder.

Embodiment 51. The method of any one of Embodiments 45 to 50, further comprising withdrawing the placement catheter from the bladder.

Embodiment 52. The method of Embodiment 51, wherein the stylet remains within the lumen of the placement catheter during the withdrawing of the placement catheter from the bladder.

Embodiment 53. A kit of parts, comprising: (i) an inserter assembly of any one of Embodiment 1 to 44; and (ii) an intravesical device suited for introduction into a patient's bladder using the inserter assembly.

Modifications and variations of the methods and devices described herein will be obvious to those skilled in the art from the foregoing detailed description. Such modifications and variations are intended to come within the scope of the appended claims.

1. An inserter assembly for deploying an intravesical device into a urinary bladder of a patient, the inserter assembly comprising:
a placement catheter defining a lumen, the lumen extending from a proximal catheter end to an exit opening;
a receiving funnel secured to the placement catheter at the proximal catheter end; and
a stylet removably received through the receiving funnel within the lumen of the placement catheter, wherein the stylet comprises (i) a stylet body extending between a proximal stylet end and a distal stylet end, the stylet body being dimensioned to drive the intravesical device through the lumen and the exit opening into the bladder, and (ii) a paddle fixed to the stylet body about the proximal stylet end, wherein the paddle is engageable with the receiving funnel.

2. The inserter assembly of claim 1, wherein the paddle is overmolded about the stylet body at the proximal stylet end, or is integral with the stylet body.

3. The inserter assembly of claim 1, wherein the paddle defines a curved thumb rest at a proximal end, and optionally further defines a curved finger rest opposing the curved thumb rest.

4. The inserter assembly of claim 1, wherein the paddle comprises a grip surface.

5. The inserter assembly of claim 1, wherein the paddle is dimensioned to be received within the receiving funnel.

6. The inserter assembly of claim 1, wherein the paddle comprises a fitting being lockably engageable with the receiving funnel to removably secure the stylet to the placement catheter.

7. The inserter assembly of claim 6, wherein the fitting is engageable with an interior surface of the receiving funnel.

8. The inserter assembly of claim 6, wherein one or both of the fitting and the receiving funnel comprises at least one clip securable to the other of the fitting and the receiving funnel.

9. The inserter assembly of claim 6, wherein the fitting defines a circumferential ridge, and wherein the receiving funnel defines a circumferential channel dimensioned to matingly engage the circumferential ridge.

10. The inserter assembly of claim 6, wherein the fitting is configured to generate audible and/or tactile feedback during engagement with the receiving funnel.

11. The inserter assembly of claim 1, wherein the receiving funnel is overmolded about the proximal catheter end, or is integral with the placement catheter at the proximal catheter end.

12. The inserter assembly of claim 1, wherein the receiving funnel has one or both of an oval profile and an oval perimeter.

13. The inserter assembly of claim 1, wherein the receiving funnel extends asymmetrically in a direction transverse to the stylet body.

14. The inserter assembly of claim 1, wherein the receiving funnel defines a funnel lumen configured to transfer lubricant from the receiving funnel to the lumen of the placement catheter, optionally wherein the receiving funnel is dimensioned to receive at least 1 mL of the lubricant.

15. The inserter assembly of claim 14, wherein the receiving funnel is shaped to facilitate transfer of the lubricant from a packet into the lumen of the placement catheter.

16. The inserter assembly of claim 1, wherein the stylet body has a solid cross-section along an entirety of the stylet body.

17. The inserter assembly of claim 1, wherein the placement catheter is transparent or translucent to visually determine the position of the intravesical device or the stylet within the lumen.

18. The inserter assembly of claim 1, wherein a distal catheter end of the placement catheter comprises (i) a rounded atraumatic tip, and/or (ii) a coudé bend, which optionally is aligned with a major axis of the retrieval funnel such that an orientation of the retrieval funnel is indicative of an orientation of the coudé bend.

19. The inserter assembly of claim 1, wherein the placement catheter comprises depth markings indicative of insertion depth and exit opening orientation.

20. The inserter assembly of claim 1, wherein the placement catheter has (i) a length of from 10 cm to 40 cm, and/or (ii) an outer diameter of from 5 Fr (1.67 mm outer diameter) to 30 Fr (10 mm outer diameter).

21. The inserter assembly of claim 1, wherein one or both of the placement catheter and the stylet include a radiomarker.

22. The inserter assembly of claim 1, wherein one or more of the placement catheter, the paddle, the receiving funnel, and the stylet are formed of the same or different polymeric material.

23. The inserter assembly of claim 1, wherein the paddle comprises:
a central body portion surrounding the proximal stylet end;
a thumb rest defined at a proximal paddle end; and
a pair of wing portions extending laterally from the central body portion between the thumb rest and a distal paddle end.

24. The inserter assembly of claim 23, wherein the thumb rest has a concave surface facing away from the central body, an oval shaped periphery, or both a concave surface facing away from the central body and an oval shaped periphery.

25. A kit of parts, comprising:
an inserter assembly of claim 1; and
an intravesical device suited for introduction into a patient's bladder using the inserter assembly.
